(19) United States
(12) Patent Application Publication
(10) Pub. No.: US 2018/0043776 A1
(43) Pub. Date: Feb. 15, 2018

(54) Breathalyzer System

(71) Applicant: Youssef Labib, Mississauga (CA)

(72) Inventor: Youssef Labib, Mississauga (CA)

(21) Appl. No.: 15/232,927

(22) Filed: Aug. 10, 2016

(51) Int. Cl. B60K 28/06; G01N 33/497

(52) U.S. Cl. CPC B60K 28/063; G01N 33/4972

(57) ABSTRACT

A breathalyzer system includes a vehicle that has an ignition. A receiver is concealed within the vehicle and the receiver is electrically coupled to the ignition. The receiver selectively actuates the ignition. A breathalyzer unit is provided. The breathalyzer unit is in electrical communication with the receiver. The breathalyzer unit may be breathed through thereby facilitating the breathalyzer unit to determine a blood alcohol concentration. The receiver actuates the ignition when the breathalyzer unit determines the blood alcohol concentration is below a legal limit. Thus, the vehicle may be driven. The receiver inhibits the ignition from being actuated when the breathalyzer unit determines the blood alcohol concentration is above the legal limit. Thus, the vehicle is inhibited from being driven.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIE THE OFFICE ELECTRONIC FILING SYSTEM

Not Applicable

### STATEMENT REGARDING PRIOR DISCLOSURES BY THE INVENTOR OR JOINT INVENTOR

Not Applicable

### (g) BACKGROUND OF THE INVENTION

### (1) Field of the Invention

### (2) Description of Related Art Including Information Disclosed Under 37 CFR 1.97 and 1.98

The disclosure and prior art relates to breathalyzer devices and more particularly pertains to a new breathalyzer device for inhibiting a vehicle from being driven when a user's blood alcohol concentration is above a legal limit.

### BRIEF SUMMARY OF THE INVENTION

An embodiment of the disclosure meets the needs presented above by generally comprising a vehicle that has an ignition. A receiver is concealed within the vehicle and the receiver is electrically coupled to the ignition. The receiver selectively actuates the ignition. A breathalyzer unit is provided. The breathalyzer unit is in electrical communication with the receiver. The breathalyzer unit may be breathed through thereby facilitating the breathalyzer unit to determine a blood alcohol concentration. The receiver actuates the ignition when the breathalyzer unit determines the blood alcohol concentration is below a legal limit. Thus, the vehicle may be driven. The receiver inhibits the ignition from being actuated when the breathalyzer unit determines the blood alcohol concentration is above the legal limit. Thus, the vehicle is inhibited from being driven.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a breathalyzer unit of a breathalyzer system according to an embodiment of the disclosure.

FIG. 2 is a front view of breathalyzer unit of an embodiment of the disclosure.

FIG. 3 is a back phantom view of a breathalyzer unit of an embodiment of the disclosure.

FIG. 4 is a perspective in-use view of an embodiment of the disclosure.

FIG. 5 is a schematic view of an embodiment of the disclosure.

### DETAILED DESCRIPTION OF THE INVENTION

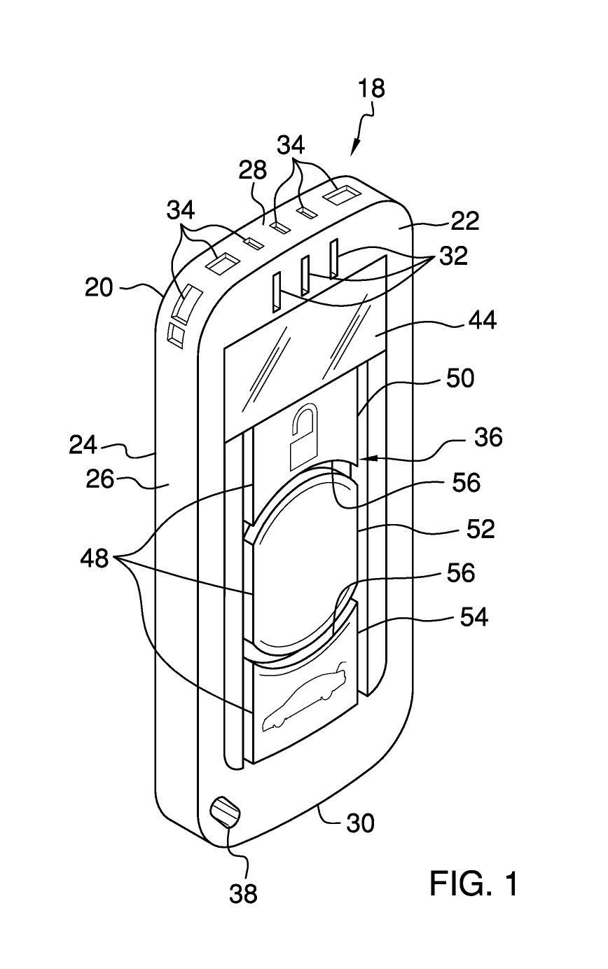

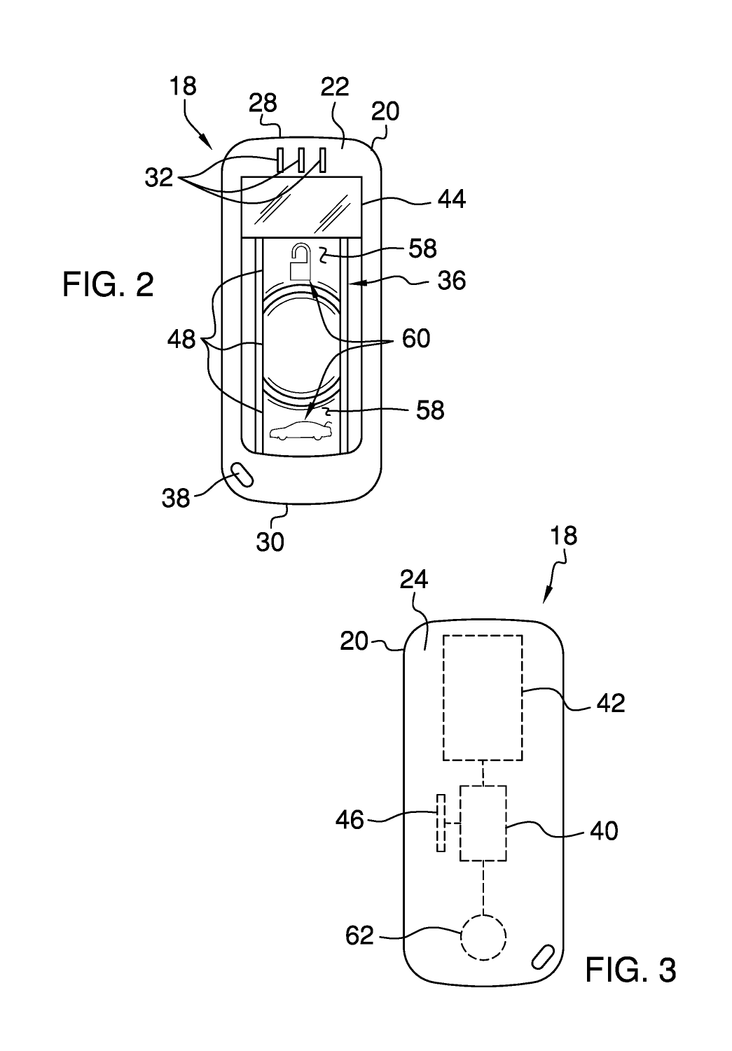

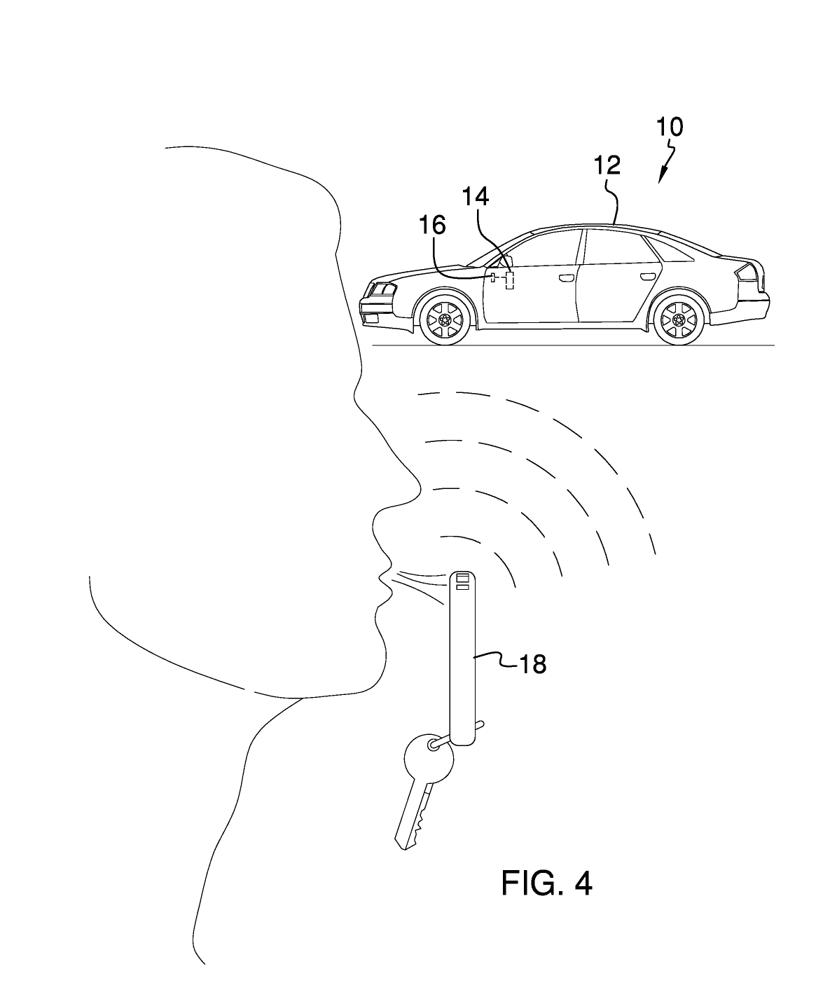

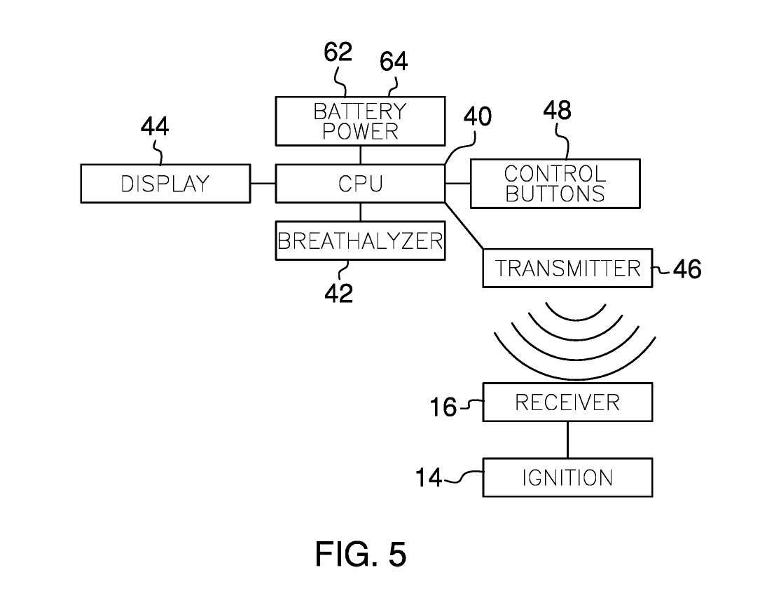

With reference now to the drawings, and in particular to FIGS. 1 through 5 thereof, a new breathalyzer device embodying the principles and concepts of an embodiment of the disclosure and generally designated by the reference numeral 10 will be described.

As best illustrated in FIGS. 1 through 5, the breathalyzer system 10 generally comprises a vehicle 12 that has an ignition 14. The vehicle 12 may be a passenger vehicle 12 or the like. A receiver 16 is provided and the receiver 16 is concealed within the vehicle 12. Thus, the receiver 16 is protected from tampering. The receiver 16 is electrically coupled to the ignition 14 and the receiver 16 selectively actuates the ignition 14. The receiver 16 may be a radio frequency receiver 16 or the like.

A breathalyzer unit 18 is provided. The breathalyzer unit 18 may be carried on a key ring or the like. The breathalyzer unit 18 is in electrical communication with the receiver 16. The breathalyzer unit 18 may be breathed through thereby facilitating the breathalyzer unit 18 to determine a blood alcohol concentration of a user.

The receiver 16 actuates the ignition 14 when the breathalyzer unit 18 determines the blood alcohol concentration is below a legal limit. Thus, the vehicle 12 may be driven. The receiver 16 inhibits the ignition 14 from being actuated when the breathalyzer unit 18 determines the blood alcohol concentration is above the legal limit. Thus, the vehicle 12 is inhibited from being driven. The legal limit may be a legal limit as determined by a state in which the vehicle 12 is registered.

The breathalyzer unit 18 comprises a housing 20 that has a front wall 22, a back wall 24 and a peripheral wall 26 extending therebetween. The peripheral wall 26 has a top side 28 and a bottom side 30. The front wall 22 has a plurality of first slots 32 extending into an interior of the housing 20. Each of the first slots 32 may facilitate breath of the user to pass into the housing 20.

The peripheral wall 26 has a plurality of second slots 34 extending into an interior of the housing 20. Each of the second slots 34 is in fluid communication with each of the first slots 32. Thus, each of the second slots 34 facilitates the breath to exit the housing 20. Each of the first slots 32 may be positioned closer to the top side 28 of the housing 20 than the bottom side 30 of the housing 20. Each of the second slots 34 may be positioned on the top side 28 of the housing 20.

The front wall 22 has a recess 36 extending toward the back wall 24. The recess 36 may extend substantially between the top side 28 and the bottom side 30. The housing 20 has an aperture 38 extending through the front wall 22 and the back wall 24. The aperture 38 may receive the key ring thereby facilitating the housing 20 to be carried.

A processor 40 is provided. The processor 40 is positioned within the housing 20. The processor 40 selectively generates a drive sequence. The processor 40 may be an electronic processor 40 or the like.

A breathalyzer 42 is provided and the breathalyzer 42 is positioned within the housing 20. The breathalyzer 42 is electrically coupled to the processor 40. The breathalyzer 42 is in fluid communication between each of the first slots 32 and each of the second slots 34. Thus, the breathalyzer 42 may determine the blood alcohol concentration of the breath. The processor 40 generates the drive sequence when the breathalyzer 42 determines the blood alcohol concentration of the breath is below the legal limit. The breathalyzer 42 may be an electronic breathalyzer 42 or the like.

A display 44 is positioned on the front wall 22 of the housing 20 and the display 44 may be observed. The display 44 is electrically coupled to the processor 40 and the display 44 may display 44 the blood alcohol concentration of the breath. The display 44 may be positioned between the recess 36 and the first slots 32. Moreover, the display 44 may be an LED display 44 or the like.

A transmitter 46 is provided and the transmitter 46 is positioned within the housing 20. The transmitter 46 is electrically coupled to the processor 40. The transmitter 46 is in electrical communication with the receiver 16. The transmitter 46 transmits the drive sequence to the receiver 16. Thus, the receiver 16 actuates the ignition 14 when the receiver 16 receives the drive sequence.

A plurality of buttons 48 is provided. Each of the buttons 48 is coupled to the front wall 22 of the housing 20 and each of the buttons 48 may be manipulated. Each of the buttons 48 is electrically coupled to the processor 40. Moreover, each of the buttons 48 controls operational parameters of the vehicle 12. Each of the buttons 48 is positioned within the recess 36.

The plurality of buttons 48 may include a first button 50, a second button 52 and a third button 54. Moreover, the plurality of buttons 48 may be vertically distributed in the recess 36. The second button 52 may have a circular shape. Each of said first button 50 and the second button 52 may have a curved edge 56. The curved edge 56 corresponding to each of the first 50 and second 52 buttons may be substantially coextensive with the second button 52.

Each of the first 50 and second 52 buttons may have an exposed surface 58. Indicia 60 may be printed on the exposed surface 58 corresponding to each of the first 50 and second 52 buttons. The indicia 60 corresponding to the first button 50 may be an image of an unlocked padlock. The indicia 60 corresponding to the third button 54 may be an image of an open trunk of a vehicle. The first button 50 may unlock doors of the vehicle 12 when the first button 50 is manipulated. The third button 54 may open a trunk of the vehicle 12 when the third button 54 is manipulated. The second button 52 may turn the breathalyzer 42 on.

A power supply 62 is provided. The power supply 62 is positioned within the housing 20. The power supply 62 is electrically coupled to the processor 40. The power supply 62 comprises at least one battery 64.

In use, the second button 52 is manipulated to turn the breathalyzer 42 on. The housing 20 is manipulated to position the first slots 32 to receive the breath. The user breathes through the first slots 32. The breathalyzer 42 determines the blood alcohol concentration of the user's breath.

The processor 40 generates the drive sequence when the blood alcohol concentration is below the legal limit. Thus, the receiver 16 actuates the ignition 14 and the vehicle 12 may be driven. The processor 40 does not generate the drive sequence when the blood alcohol concentration is above the legal limit. Thus, the receiver 16 does not actuate the ignition 14 and the vehicle 12 is inhibited from being driven.

With respect to the above description then, it is to be realized that the optimum dimensional relationships for the parts of an embodiment enabled by the disclosure, to include variations in size, materials, shape, form, function and manner of operation, system and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A breathalyzer system being configured to inhibit an intoxicated driver from driving a vehicle, said system comprising:
a vehicle having an ignition;
a receiver being concealed within said vehicle wherein said receiver is configured to be protected from tampering, said receiver being electrically coupled to said ignition, said receiver selectively actuating said ignition; and
a breathalyzer unit being configured to be carried, said breathalyzer unit being in electrical communication with said receiver, said breathalyzer unit being configured to be breathed through thereby facilitating said breathalyzer unit to determine a blood alcohol concentration, said receiver actuating said ignition when said breathalyzer unit determines the blood alcohol concentration is below a legal limit wherein said vehicle is configured to be driven, said receiver inhibiting said ignition from being actuated when said breathalyzer unit determines the blood alcohol concentration is above the legal limit wherein said vehicle is configured to be inhibited from being driven, said breathalyzer unit including a housing;
an aperture extending through said housing;
a key ring extending through said aperture wherein said key ring is coupled to said breathalyzer unit; and
a display being positioned on said housing wherein said display is configured to be observed, said display being operationally coupled to said breathalyzer unit to display the blood alcohol concentration of the breath.

2. The system according to claim 1, further comprising said housing having a front wall, a back wall and a peripheral wall extending therebetween, said front wall having a plurality of first slots extending into an interior of said housing wherein each of said first slots is configured to facilitate breath to pass into said housing, said peripheral wall having a plurality of second slots extending into an interior of said housing, each of said second slots being in fluid communication with each of said first slots wherein each of said second slots is configured to facilitate the breath to exit said housing.

3. (canceled)

4. The system according to claim 2, further comprising a processor being positioned within said housing, said processor selectively generating a drive sequence.

5. The system according to claim 4, further comprising a breathalyzer being positioned within said housing, said breathalyzer being electrically coupled to said processor, said breathalyzer being in fluid communication between each of said first slots and each of said second slots wherein said breathalyzer is configured to determine the blood alcohol concentration of the breath, said processor generating said drive sequence when said breathalyzer determines the blood alcohol concentration of the breath is below the legal limit.

6. The system according to claim 4, further comprising said display being positioned on said front wall of said housing wherein said display is configured to be observed, said display being electrically coupled to said processor wherein said display is configured to display the blood alcohol concentration of the breath.

7. The system according to claim 4, further comprising a transmitter being positioned within said housing, said transmitter being electrically coupled to said processor, said transmitter being in electrical communication with said receiver, said transmitter transmitting said drive sequence to said receiver, said receiver actuating said ignition when said receiver receives said drive sequence.

8. The system according to claim 4, further comprising:
said housing having a recess; and
a plurality of buttons, each of said buttons being coupled to said front wall of said housing wherein each of said buttons is configured to be manipulated, each of said buttons being electrically coupled to said processor, each of said buttons controlling operational parameters of said vehicle, each of said buttons being positioned within said recess.

9. The system according to claim 4, further comprising a power supply being positioned within said housing, said power supply being electrically coupled to said processor, said power supply comprising at least one battery.

10. A breathalyzer system being configured to inhibit an intoxicated driver from driving a vehicle, said system comprising:
a vehicle having an ignition;
a receiver being concealed within said vehicle wherein said receiver is configured to be protected from tampering, said receiver being electrically coupled to said ignition, said receiver selectively actuating said ignition; and
a breathalyzer unit being configured to be carried, said breathalyzer unit being in electrical communication with said receiver, said breathalyzer unit being configured to be breathed through thereby facilitating said breathalyzer unit to determine a blood alcohol concentration, said receiver actuating said ignition when said breathalyzer unit determines the blood alcohol concentration is below a legal limit wherein said vehicle is configured to be driven, said receiver inhibiting said ignition from being actuated when said breathalyzer unit determines the blood alcohol concentration is above the legal limit wherein said vehicle is configured to be inhibited from being driven, said breathalyzer unit comprising:
a housing having a front wall, a back wall and a peripheral wall extending therebetween, said front wall having a plurality of first slots extending into an interior of said housing wherein each of said first slots is configured to facilitate breath to pass into said housing, said peripheral wall having a plurality of second slots extending into an interior of said housing, each of said second slots being in fluid communication with each of said first slots wherein each of said second slots is configured to facilitate the breath to exit said housing, said front wall having a recess extending toward said back wall, said housing having an aperture extending through said front wall and said back wall wherein said aperture is configured to receive a key ring thereby facilitating said housing to be carried,
a processor being positioned within said housing, said processor selectively generating a drive sequence,
a breathalyzer being positioned within said housing, said breathalyzer being electrically coupled to said processor, said breathalyzer being in fluid communication between each of said first slots and each of said second slots wherein said breathalyzer is configured to determine the blood alcohol concentration of the breath, said processor generating said drive sequence when said breathalyzer determines the blood alcohol concentration of the breath is below the legal limit,
a display being positioned on said front wall of said housing wherein said display is configured to be observed, said display being electrically coupled to said processor wherein said display is configured to display the blood alcohol concentration of the breath,
a transmitter being positioned within said housing, said transmitter being electrically coupled to said processor, said transmitter being in electrical communication with said receiver, said transmitter transmitting said drive sequence to said receiver, said receiver actuating said ignition when said receiver receives said drive sequence,
a plurality of buttons, each of said buttons being coupled to said front wall of said housing wherein each of said buttons is configured to be manipulated, each of said buttons being electrically coupled to said processor, each of said buttons controlling operational parameters of said vehicle, each of said buttons being positioned within said recess,
a power supply being positioned within said housing, said power supply being electrically coupled to said processor, said power supply comprising at least one battery.
